SEQUENCE LISTING (2 sequences)

SEQ ID NO: 1 - DNA, 1539 bp, Herpes simplex virus
atggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttag

SEQ ID NO: 2 - protein, 512 aa, Herpes simplex virus
MATDIDMLIDLGLDLSDSDLDEDPPEPAESRRDDLESDSNGECSSSDEDMEDPHGEDGPEPILDAARPAVRPSRPEDPGVPSTQTPRPTERQGPNDPQPAPHSVWSRLGARRPSCSPERHGGKVARLQPPPTKAQPARGGRRGRRRGRGRGGPGAADGLSDPRRRAPRTNRNPGGPRPGAGWTDGPGAPHGEAWRGSEQPDPPGGPRTRSVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTAAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

(19) United States
(12) Patent Application Publication
(10) Pub. No.: US 2005/0226847 A1
(43) Pub. Date: Oct. 13, 2005

(54) Adeno-associated virus producer system

(76) Inventors: Robert Stuart Coffin, Oxfordshire (GB); Matthew James Booth, London (GB)

Correspondence Address: FULBRIGHT & JAWORSKI, LLP, 1301 MCKINNEY, SUITE 5100, HOUSTON, TX, 77010-3095, US

(21) Appl. No.: 10/510,528

(22) PCT Filed: Apr. 11, 2003
(86) PCT No.: PCT/GB03/01585

§ 371 Date: Oct. 7, 2004

(30) Foreign Application Priority Data
Apr. 11, 2002 (DE) 0208390.5

(51) Int. Cl. A61K 48/00; C12N 7/00; C12N 15/861

(52) U.S. Cl. 424/93.2; 435/456; 435/235.1

(57) ABSTRACT

The present invention provides the use of a replication competent herpes virus which (a) lacks a functional wild-type HSV ICP27 gene; and (b) comprises an ICP27 gene encoding an ICP27 protein which allows replication of said herpes virus to occur and which has a reduced ability to inhibit RNA splicing compared to wild-type HSV ICP27 in the production of an adeno-associated virus (AAV) vector.

### CROSS-REFERENCE TO RELATED APPLICATIONS

This application is a national stage application of PCT/GB03/01585 filed Apr. 11, 2003, claiming priority to GB 0208390.5 filed Apr. 11, 2002.

### TECHNICAL FIELD

The present invention relates to the use of a herpes helper virus in the production of adeno-associated virus (AAV) vectors, to a novel herpes helper virus and to an improved method of producing AAV vectors using a herpes helper virus.

### BACKGROUND OF THE INVENTION

Adeno-associated virus (AAV) vectors are promising for gene delivery and gene therapy in a number of target tissues including muscle, liver and brain. AAV is a naturally defective virus which requires a helper adenovirus or herpes virus for growth. AAV vectors are versions of AAV in which the genes encoding the necessary replication (rep) and structural (cap) proteins have been deleted to allow insertion of the sequences to be delivered between the remaining terminal repeat sequences. For growth of vectors therefore, not only is a helper virus required, but the genes encoding the rep and cap proteins must be delivered to or be present in the cells used for production to provide the rep and cap functions in trans.

Such production systems have provided a problem in the field because stable cell lines expressing appropriate levels of rep and cap genes have been difficult to produce. Versions of helper adenovirus containing these genes have also not solved the problem. Thus in general for the production of AAV vectors, a plasmid encoding the rep and cap genes and a plasmid containing the DNA sequence to be packaged flanked by AAV terminal repeats are transfected into cells and then the cells infected with a helper adenovirus. This process for the production of AAV vectors is relatively laborious and inefficient and is hard to scale up.

AAV production systems using herpes simplex virus (HSV) as a helper virus are also known. An amplicon system has been described but the use of HSV as a helper virus for AAV growth has to date been best accomplished using non-replicating mutants of HSV deleted for ICP27 (Conway et al 1999).

### BRIEF SUMMARY OF THE INVENTION

The present inventors have devised a novel AAV production system which uses as a helper virus a herpes virus, preferably a herpes simplex virus (HSV), having improved properties compared to previous herpes helper viruses. When HSV is used, the novel AAV production system of the present invention uses a HSV helper virus containing an ICP27 protein which allows HSV replication to occur but which shows reduced inhibition of mRNA splicing compared to wild-type HSV-1 ICP27. The ICP27 protein may be a mutant protein which is mutated so that the usual inhibition of mRNA splicing associated with expression of ICP27 is reduced or prevented, but so that virus replication is not prevented. Alternatively, a non-HSV homologue of ICP27 with these properties may be used in the producer system of the invention.

HSV genes are in the main non-spliced while the majority of mammalian cellular genes are spliced. HSV has evolved a mechanism of enhancing the expression of HSV genes as compared to host genes by inhibiting mRNA splicing. This effect is largely mediated through the HSV ICP27 protein, a multi-functional essential gene required for virus growth and replication. The AAV rep and cap genes are multiply spliced and this splicing will therefore be inhibited in the presence of wild type HSV.

The use of a herpes helper virus capable of replicating and having a reduced property of inhibiting the splicing of mRNA in infected cells can enable an increased efficiency of production of AAV vectors to be achieved compared to the use of non-replicative HSV helper viruses which have been found to be most effective previously. This AAV vector production process may be easily scaled up and is thus useful for producing AAV vectors on an industrial scale.

Accordingly, the present invention provides:
the use of a replication competent herpes virus which
(a) lacks a functional wild-type HSV ICP27 gene; and
(b) comprises a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27
   in the production of an adeno-associated virus (AAV) vector;
a replication competent herpes virus which
   (a) lacks a functional wild-type HSV ICP27 gene;
   (b) comprises a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27; and
   (c) comprises AAV rep and/or cap genes and/or an AAV vector sequence;
a method of producing an AAV vector comprising:
   (i) introducing into producer cells:
   (a) a herpes virus which lacks a functional wild-type HSV ICP27 gene;
   (b) a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27;
   (c) AAV rep and cap genes; and
   (d) an AAV vector sequence; and
   (ii) isolating the AAV vector particles produced;
an AAV vector produced by a method of the invention;
a pharmaceutical composition comprising an AAV vector according to the invention and a pharmaceutically acceptable carrier or diluent;
a method of producing a pharmaceutical composition comprising formulating an AAV vector according to the invention with a pharmaceutically acceptable carrier or diluent;
a method of gene therapy comprising administering a therapeutically effective amount of an AAV vector according to the invention to a patient in need thereof; and
a kit for producing an AAV vector comprising:
   (a) a replication competent herpes virus which lacks a functional wild-type HSV ICP27 gene;
   (b) a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27;
   (c) AAV rep and cap genes;
   (d) an AAV vector sequence; and optionally
   (e) producer cells

wherein said nucleic acid (b), said AAV rep and cap genes (c) and/or said AAV vector sequence (d) are incorporated into said herpes virus (a), are present on separate plasmids or are stably integrated into said producer cells (e).

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE SEQUENCE LISTING

SEQ ID No. 1 is the nucleotide sequence encoding ICP27 from HSVI (GenBank Accession No. AF220940).

SEQ ID No. 2 is the amino acid sequence of ICP27 from HSV1 (GenBank Accession No. AF220940).

### DETAILED DESCRIPTION OF THE INVENTION

The invention provides the use of a replication competent herpes virus which
(a) lacks a functional wild-type HSV ICP27 gene; and
(b) comprises a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27

in the production of an adeno-associated virus (AAV) vector.

A herpes virus which lacks a functional wild-type HSV ICP27 may be an HSV, preferably HSV-1, HSV-2 or an intertypic recombinant HSV, in which the wild-type HSV ICP27 gene has been deleted or in which the HSV ICP27 gene has been mutated such that one or more of the normal functions of the ICP27 protein is inhibited or prevented. Alternatively the herpes virus may be a non-HSV herpes virus which naturally does not contain a wild-type HSV ICP27 gene or a non-HSV herpes virus which lacks a functional wild-type HSV ICP27 homologue.

By a A functional equivalent@ it is meant a herpes virus gene that performs the same or a similar function as ICP27 in HSV and that exhibits sequence homology, at the amino acid level, to the HSV ICP27 protein. Typically, a functional equivalent of, the HSV ICP27 gene will show at least 15%, preferably at least 20%, more preferably at least 50%, at least 70%, at least 80%, at least 90%, at least 95% or at least 98% sequence identity at the amino acid level to the HSV ICP27 protein sequence shown in SEQ ID No. 2 over a region of at least 20, preferably at least 30, for instance at least 40, 60 or 100 or more contiguous amino acids, more preferably over the whole length of SEQ ID No. 2.

Methods of measuring protein and nucleotide homology are well known in the art and it will be understood by those of skill in the art that in the present context, protein homology is calculated on the basis of amino acid identity (sometimes referred to as “hard homology”).

Methods of measuring nucleic acid and protein homology are well known in the art. For example the UWGCG Package provides the BESTFIT program which can be used to calculate homology (for example used on its default settings) (Devereux et al. (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al. (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al., 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Homologues of the HSV ICP27 gene from other herpes viruses can be identified in a number of ways, for example by probing genomic or cDNA libraries made from the herpes virus with probes comprising all or part of the sequence shown in SEQ ID No. 1 under conditions of medium to high stringency (0.2× SSC/0.1% SDS at from about 40° C. to about 55° C.). Alternatively, species homologues may also be obtained using degenerate PCR which will use primers designed to target sequences within the homologues encoding conserved amino acid sequences. The primers will contain one or more degenerate positions and will be used at stringency conditions lower than those used for cloning sequences with single sequence primers against known sequences (for example, 2×SSC at 60° C.).

A homologue of the HSV ICP27 gene from a non-HSV herpes virus is typically a functional equivalent thereof. This can be confirmed by testing the functional properties of the homologue. Generally, a functional equivalent of HSV ICP27 is an immediate early gene and encodes a protein which is capable of inhibiting gene splicing, which is essential for efficient herpes virus replication. Such functional equivalents include the equine herpes virus 1 UL3 gene, the VZV ORF4 gene and the HHV8 ORF57 gene.

The wild-type HSV ICP27 gene, or functional equivalent thereof in a non-HSV herpes virus, may be rendered functionally inactive by several techniques well known in the art. For example, they may be rendered functionally inactive by deletion(s), substitution(s) or insertion(s), preferably by deletion. Deletions may remove portions of the genes or the entire gene. For example, deletion of only one nucleotide may be made, resulting in a frame shift. However, preferably larger deletions are made, for example at least 25%, more preferably at least 50% of the total coding and non-coding sequence (or alternatively, in absolute terms, at least 10 nucleotides, more preferably at least 100 nucleotides, most preferably, at least 1000 nucleotides). It is particularly preferred to remove the entire gene and some of the flanking sequences. Inserted sequences may include the nucleic acid encoding an ICP27 protein or functional equivalent thereof having impaired RNA splicing inhibitory activity but which allows herpes virus replication to occur.

Mutations may be made in the herpes viruses by homologous recombination methods well known to those skilled in the art. For example, HSV genomic DNA is transfected together with a vector, preferably a plasmid vector, comprising the mutated sequence flanked by homologous HSV sequences. The mutated sequence may comprise deletion(s), insertion(s) or substitution(s), all of which may be constructed by routine techniques. Insertions may include selectable marker genes, for example lacZ, for screening recombinant viruses by, for example, β-galactosidase activity.

The nucleic acid encoding an ICP27 protein which protein allows herpes virus replication to occur and which has a reduced ability to inhibit RNA splicing compared to wild-type HSV ICP27, or functional equivalent thereof from a non-HSV herpes virus is preferably operably linked to promoter sequence permitting expression of the ICP27 protein in producer cells. Any suitable promoter sequence may be used, for example an immediate early gene promoter. Preferably the promoter sequence is an ICP27 promoter, such as an HSV-1 or HSV-2 ICP27 promoter. More preferably, the promoter is the endogenous ICP27 promoter in HSV or the endogenous promoter of an ICP27 homologue in a non-HSV herpes virus. Alternatively, the promoter may be a mammalian promoter, such as a promoter of mammalian or viral origin active in mammalian cells. Examples of suitable mammalian promoters include the CMV, RSV, MMLV or SV40 promoters.

The ICP27 protein in the herpes virus is typically an HSV ICP27 protein which contains a mutation that prevents the ICP27 protein from inhibiting RNA splicing or which reduces the level of inhibition of RNA splicing compared to wild-type HSV ICP27 protein. The mutation of the ICP27 protein does not prevent replication of the herpes virus. However, replication of the herpes virus may be less efficient than replication of the parent herpes virus which comprises a wild-type ICP27 gene, or functional equivalent thereof. Herpes virus replication may be monitored by any suitable method, for example by infecting host cells with the herpes virus comprising the ICP protein, or functional equivalent thereof, and determining the virus titre after a period of incubation, for example for 24 to 48 hours.

A herpes helper virus of the invention has a reduced property of inhibiting the splicing of mRNA in infected cells. Inhibition of RNA splicing by the ICP27 protein may be reduced by 10, 20, 30 or 40%, preferably by 50, 60, 70, 80 or 90% compared to wild-type HSV ICP27 protein, for example to wild-type HSV-1 or HSV-2 ICP27 protein. Any suitable assay for monitoring RNA splicing may be used to determine whether an ICP27 protein has a reduced ability to inhibit RNA splicing. For example, RNA splicing may be monitored by northern blotting or RNase protection assays to determine the relative levels of spliced and unspliced transcripts present. Typically RNA splicing assays are carried out both in the presence and absence of wild-type HSV ICP27 and in the presence and absence of the ICP27 protein, or functional equivalent thereof being tested. RNA splicing in the presence of the wild-type HSV1 ICP27 protein may be compared to RNA splicing in the presence of the ICP27 protein, or functional equivalent thereof, being tested to determine whether the test protein has a reduced ability to inhibit RNA splicing.

A herpes helper virus of the invention thus allows more mRNA splicing to occur in infected cells than can occur in cells infected with a herpes virus comprising a wild-type HSV ICP27 gene. Preferably the herpes virus of the invention allows a significant amount of mRNA splicing to occur in infected cells, i.e. the inhibition of gene splicing the ICP27 protein encoded by the herpes helper virus is negligible. RNA splicing in cells infected with a virus of the invention may be uninhibited or be inhibited by 1, 5, 10, 20, 30, 40, 50, 60, 70 or 80% compared to RNA splicing in cells not infected with a herpes virus.

The nucleic acid encoding the ICP27 protein may be mutated so that its ability to inhibit gene splicing is impaired by any technique well known in the art, for example, by deletion(s), substitution(s) or insertion(s) into the portion(s) of the gene important for the inhibition of gene splicing. It is not desirable to delete large portions of the ICP27 gene because it is necessary that the gene retains its ability to enable herpes virus replication to occur. However, small deletions, insertions and/or substitutions may be made as appropriate to diminish the gene splicing-inhibitory activity. Examples of mutant ICP27 proteins are described in Soliman et al. 1997.

Typically, the ICP27 mutant protein may have one or more mutation in the KH3 domain and/or the SM motif. Preferred mutant ICP27 have one or more mutation in the region from amino acids 400 to 512 of SEQ ID No. 2, such as between amino acids 450 and 490, amino acids 470 and 500 or amino acids 480 and 500. The mutation may be a C-terminal truncation of the ICP27 protein sequence shown in SEQ ID No. 2. More preferably, the amino acid at position 480 of SEQ ID No. 2 is mutated. The amino acids at positions 334, 487 and/or 496 of SEQ ID NO: 2 may alternatively or additionally be mutated. For example, the ICP27 protein may comprise an R480H substitution and an L334S substitution or an R480H substitution and a V487I substitution. Most preferably, the ICP27 protein is the double mutant R480H/ V496I.

Alternatively, the ICP27 protein may be a functional equivalent of HSV ICP27 from a non-HSV herpes virus, which functional equivalent does not inhibit RNA splicing, or shows reduced inhibition of RNA splicing compared to wild-type HSV ICP27 protein, and which does not prevent the herpes virus from replicating. The functional equivalent may be a wild-type (native) protein from a non-HSV herpes virus or may be a mutant protein. Where the functional equivalent is a mutant protein the mutation typically corresponds to one of the mutations mentioned herein with respect to the HSV ICP27 protein. Corresponding mutations may be determined by aligning the amino acid sequence of the functional equivalent to that of SEQ ID No. 2 using any suitable alignment technique.

The herpes virus may additionally comprise AAV rep and/or cap genes. The rep and/or cap genes are preferably inserted into a site or sites of the herpes virus genome such that replication of the herpes virus is not prevented. Suitable sites of insertion include the UL43, US5 and LAT loci but other insertion sites may also be used. The rep and cap genes comprise the coding sequences of the rep and cap genes from an AAV operably linked to control sequences. Any suitable control sequences may be used. For example, the rep and cap genes may be under the control of herpes virus promoters. Preferably, the control sequences are the promoters which usually control rep and cap expression in AAV.

The herpes virus may comprise an AAV vector sequence. The AAV vector sequence is preferably inserted into a site or sites of the herpes virus genome such that replication of the herpes virus is not prevented. Suitable sites of insertion include the UL43, US5 and LAT loci but many other insertion sites may also be used.

The AAV vector sequence is typically a DNA sequence to be packaged flanked by AAV terminal repeats. The DNA sequence to be packaged is generally an expression cassette comprising a sequence encoding a heterologous protein operably linked to a control sequence. It is preferred that the heterologous protein is a therapeutic protein.

The invention also provides a herpes virus which

(a) lacks a functional wild-type HSV ICP27 gene;

(b) comprises a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27;

(c) an AAV cap gene and/or an AAV rep gene; and/or

(d) an AAV vector sequence.

A herpes virus of the invention is preferably HSV-1 or HSV-2 or an intertypic recombinant virus containing DNA from HSV-1 and HSV-2 strains. A herpes virus of the invention may be a non-HSV virus which further lacks a functional wild-type HSV ICP27 homologue. The nucleic acid (b) and the AAV sequences (c) and (d) are as defined above.

A further aspect provided by he present invention is a method of producing an AAV vector comprising:
(i) introducing into producer cells:
(a) a herpes virus which lacks a functional wild-type HSV ICP27 gene;
(b) a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27;
(c) AAV rep and cap genes; and
(d) an AAV vector sequence; and
(ii) isolating the AAV vector particles produced.

The nucleic acid encoding an ICP27 protein (b) is as defined above. The nucleic acid may be incorporated into the herpes virus (a). Alternatively, the nucleic acid is not incorporated into the herpes virus but is present in the producer cells such that it can complement the lack of HSV ICP27 gene in the herpes virus in trans. The nucleic acid may be introduced into the producer cells by transient transfection of an plasmid into the producer cells either before or after the cells are infected with HSV. In a preferred embodiment of the invention the nucleic acid is stably integrated into the genome of the producer cells. Suitable methods of transient and stable transfection are well known in the art.

Where the nucleic acid encoding the ICP27 protein is introduced into the producer cells by transient or stable transfection, there is preferably no overlap between the nucleotide sequences in the plasmid or cell line and the nucleotide sequences of the herpes virus, thus preventing homologous recombination. Prevention of homologous recombination is important in order to prevent the generation of a herpes virus which can replicate in the absence of the producer cells. The AAV particles produced by such a method will therefore be free from any contaminating replication competent herpes virus.

The AAV rep and/or cap genes (c) and/or the AAV vector sequence (d) may be incorporated into the herpes virus (a). The rep and/or cap genes and/or AAV vector sequences are preferably inserted into a site or sites of the herpes virus genome such that replication of the herpes virus is not prevented. Suitable sites of insertion include the UL43, US5 and LAT loci but many other insertion sites may also be used.

Alternatively the AAV rep and/or cap genes (c) and/or the AAV vector sequence (d) may each be introduced into the cells by transient or stable transfection of suitable plasmids. Suitable methods of transfection are well known in the art. When transient transfection is used to introduce the AAV rep and/or cap genes and/or the AAV vector sequence into the producer cells, the transfection may be carried out before or after infection of the cells with the herpes virus. If stable transfection is used, it is preferable that the AAV rep and/or cap genes and/or AAV vector sequences are integrated into the producer cell genome prior to infection of the producer cells with the herpes virus.

Producer cells used in the invention include any cell line that supports the growth of the herpes virus. A suitable cell line is a cell line which hosts herpes viruses and forms colonies. Typically the cell line is a mammalian cell line such as a rodent or human cell line. A particularly preferred cell line is based on BHK or Vero cells.

In a further aspect, the present invention provides a kit for producing an AAV vector comprising:
(a) a replication competent herpes virus which lacks a functional wild-type HSV ICP27 gene;
(b) a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27;
(c) AAV rep and cap genes;
(d) an AAV vector sequence; and optionally
(e) producer cells

wherein (b), (c) and/or (d) are incorporated into said herpes virus (a), are present on separate plasmids or are stably integrated into said producer cells (e).

In a further aspect of the invention is provided an AAV vector produced by a method of the invention, produced using a herpes virus of the invention or using a kit of the invention. The AAV vector is typically lacking rep and cap genes and comprises a heterologous gene, i.e. a sequence encoding a heterologous protein, operably linked to control sequences. The heterologous protein is preferably a therapeutic protein. A therapeutic protein is defined herein as being a protein which it is desirable to introduce into a cell or cells of a patient in order to treat a disease or disorder.

An AAV vector of the invention may be formulated with a pharmaceutically acceptable carrier or diluent and/or may be administered to a patient in a method of treatment of a disease or disorder, for example by gene therapy.

Accordingly, the present invention provides a pharmaceutical composition for use in a method of treatment such as gene therapy comprising an AAV vector according to the invention and a pharmaceutically acceptable carrier or diluent. A method of producing a pharmaceutical composition by admixing an AAV vector of the invention with a pharmaceutically acceptable carrier or diluent is also provided.

A method of treatment of the human or animal body, for example a method of gene therapy, comprising administering a therapeutically effective amount of an AAV vector of the invention to a patient in need thereof. A therapeutically effective amount of an AAV vector is an amount which enables a therapeutic gene in the vector to be expressed in cells of a patient at a level that alleviates the symptoms of the disease or disorder to be treated or that improves the condition of the patient. A patient in need of treatment is a patient suffering from or predisposed to a disease or disorder that can be treated by administration of a therapeutic gene incorporated into an AAV vector.

The invention is illustrated by the following preferred embodiments:

Embodiment 1

(i) The HSV helper virus has the wild type HSV ICP27 gene replaced with a mutant version of ICP27 with which the inhibition of splicing is reduced but which can still support the replication of HSV. Examples of such mutations have been reported previously (Soliman et al. 1997). Alternatively the wild type ICP27 gene is replaced with a non-HSV homologue of ICP27 which supports virus growth but which displays a reduced inhibition of splicing as compared to HSV ICP27;

(ii) Plasmids encoding the AAV vector sequences and the AAV rep and cap genes are stably or transiently transfected into cells which can support the growth of HSV. In the case of stable transfection, these cells are then infected with helper viruses as described in (i) above. In the case of transient transfection, cells are infected with the helper virus before, at the same time as, or after transfection. Experiment will determine the optimal timing for this;

(iii) AAV vector particles are produced.

Embodiment 2

(i) The HSV helper virus has the wild type HSV ICP27 gene replaced with a mutant version of ICP27 with which the inhibition of splicing is reduced but which can still support the growth of HSV. Examples of such mutations have been reported previously (Soliman et al. 1997). Alternatively the wild type ICP27 gene is replaced with a non-HSV homologue of ICP27 which supports virus growth but which displays a reduced inhibition of splicing as compared to HSV ICP27;

(ii) Any of the AAV rep, cap or vector sequences to be packaged are incorporated into the helper virus described above, reducing the number of plasmids required to be stably or transiently transfected into the producer cells, prior to use for AAV vector production; and

(iii) AAV vector particles are produced

In a particularly preferred version of embodiment 2, the helper virus is an HSV in which the wild-type HSV ICP27 gene has been replaced with a mutant HSV-1 ICP27 gene. The mutant HSV-1 ICP27 gene is capable of supporting virus growth but shows a reduced inhibition of splicing compared to wild type HSV-1 ICP27 is constructed. The mutant HSV-1 ICP27 gene may be a mutant as described in Soliman et al. 1997. The rep and cap encoding sequences from AAV are inserted into the HSV helper virus. The site or sites of insertion of the rep and cap genes is chosen so that HSV replication is not prevented. Suitable sites include the UL43, US5 or LAT loci, but many others are possible. The HSV helper virus is used to infect cells, such as BHK or Vero cells, which support the growth of HSV in which the AAV vector sequence is stably integrated into the cellular genomic DNA. AAV vector particles are then produced.

In a second preferred version of embodiment 2, the HSV helper virus is identical to that used in the first preferred version except that in addition to the rep and cap encoding sequences from AAV, the AAV vector sequence is inserted into the HSV helper virus. The site of insertion of the AAV vector sequence is chosen so that HSV replication is not prevented. Suitable sites include the UL43, US5 or LAT loci, but many others are possible. The HSV helper virus is used to infect cells, such as BHK or Vero cells, which support the growth of HSV. AAV vector particles are then produced.

Embodiment 3

(i) The HSV helper virus has the wild type HSV ICP27 gene mutated such that no replication is possible in cells which do not complement the deficiency.

(ii) The cells used for production contain a version of ICP27 with which the inhibition of splicing is reduced but which can still support the growth of HSV introduced by stable or transient transfection. Alternatively a non-HSV homologue of ICP27 which supports virus growth but which displays a reduced inhibition of splicing as compared to HSV ICP27 is used;

(iii) AAV rep and cap and vector sequences are introduced and helper virus provided as in Embodiments 1 or 2 above; and

(iv) AAV vector particles are produced.

The following Examples illustrate the invention.

### Example 1

### Virus Construction

Six viruses were constructed in order to test the hypothesis that use of a virus with a mutated ICP27 gene in which inhibition of splicing was reduced was more effective at producing AAV than HSV with wild type ICP27 or HSV with ICP27 deleted.

1. 17+/UL43/repcap

This virus contains the AAV rep and cap genes inserted into the UL43 gene of wild type HSV1 strain 17+. UL43 is a non-essential HSV gene the mutation of which does not affect replication (Maclean et al). The virus was constructed by inserting the repcap gene excised from pdeltaBal (supplied by Dr Adrian Thrasher, University College London) into the unique NsiI site of the UL43 gene.

2. 17+/US5/AAVGFP

This virus contains the AAV ITRs flanking a CMV-GFP-bGH polyA cassette inserted into the US5 gene of wild type HSV1 strain 17+. US5 is a non-essential HSV gene the mutation of which does not affect replication (Balan et al).

3. 17+mut.27/UL43/repcap

This virus contains the AAV rep and cap genes inserted into the UL43 gene of a derivative of HSV1 strain 17+ in which the ICP27 gene has been mutated to include the R480H/V496I double mutation described in Soliman et al. This mutation has previously been shown to allow improved splicing as compared to wild type ICP27 (Soliman et al). The virus was constructed by inserting the repcap gene excised from pdeltaBal (supplied by Dr Adrian Thrasher, University College London) into the unique NsiI site of the UL43 gene of a virus in which the ICP27 gene had been replaced with a mutant gene in which a double point mutation (R480H/V4961; Soliman et al) had been inserted using the Stratagene Quickchange XL kit.

4. 17+mut.27/US5/AAVGFP

This virus contains the AAV ITRs flanking a CMV-GFP-bGH polyA cassette inserted into the US5 gene of HSV1 strain 17+in which the ICP27 gene has been mutated according to Soliman et al, as above.

5. 17+27-/UL43/repcap

This virus contains the AAV rep and cap genes inserted into the UL43 gene of a derivative of HSV1 strain 17+ in which the ICP27 gene has been deleted. The virus was constructed by inserting the repcap gene excised from pdeltaBal (supplied by Dr Adrian Thrasher, University College London) into the unique NsiI site of the UL43 gene of 17+27-.

6. 17+27-/US5/AAVGFP

This virus contains the AAV ITRs flanking a CMV-GFP-bGH polyA cassette inserted into the US5 gene of HSV1 strain 17+ in which the ICP27 gene has been deleted, as above.

### Example 2

### Production of AAV

Viruses 1 & 2, 3 & 4, or 5 & 6 were tested for AAV production using the following procedure:

1. BHK or B130 cells (Howard et al) (175 cm² flask) were infected with the appropriate pair of viruses at a range of MOIs and incubated at 37° C. for 2 days. B130 cells are BHK cell which contain the HSV1 ICP27 gene allowing the growth of HSV1 deleted for ICP27 (Howard et al Gene Ther. 1998 Aug: 5(8):1137-47.)

2. Cells were harvested 2 days p.i. using a cell scraper, transferred to a 50 ml Falcon tube and spun at 1000 rpm for 10 min.

3. The supernatant was removed and the pellet resuspended in 1 ml TMN buffer (50 mM Tris pH 8.0, 5 mM MgCl₂, 0.15M NaCI).

4. Three cycles of freeze thawing was carried out using dry ice and a 37° C. water bath, vortexing thoroughly between cycles.

5. 50U benzonase was then added to the lysate and incubated for 30 min at 37° C.

6. The lysate was clarified by centrifugation at 400 rpm for 20 min and the supernatant containing AAV was removed and stored at −20° C.

7. AAV preparations were titred for capsid number using Progen's AAV titration ELISA kit (Cat. No. PRATV)

The results of this experiment are set out in the Table 1 below.

TABLE 1
AAV production using different HSV viruses

| | | | | AAV Capsid |
|---|---|---|---|---|
| Virus | Virus | | Cell | titre |
| (a) | (b) | MOI used | type | (particles/ml) |
| 17+/UL43/repcap | 17+/US5/AAVGFP | 0.5 | BHK | 1.2 × 10⁶ |
| | | 0.05 | | 2.4 × 10⁸ |
| | | 0.005 | | 1.7 × 10⁸ |
| 17+mut.27/UL43/ | 17+mut.27/US5/ | 0.5 | BHK | 1.9 × 10¹⁰ |
| repcap | AAVGFP | 0.05 | | 1.8 × 10¹⁰ |
| | | 0.005 | | 1.8 × 10¹⁰ |
| 17+27−/UL43/ | 17+27−/US5/ | 5 | BHK | 2.0 × 10¹⁰ |
| repcap | AAVGFP | 0.5 | | 2.1 × 10¹⁰ |
| | | 0.05 | | 8.0 × 10⁹ |
| | | 0.005 | | 6.2 × 10⁶ |
| 17+27−/UL43/ | 17+27−/US5/ | 5 | B130 | 6 × 10⁸ |
| repcap | AAVGFP | 0.5 | | 5.6 × 10⁸ |
| | | 0.05 | | 6.2 × 10⁸ |
| | | 0.005 | | 1.1 × 10⁸ |

This shows that while all three sets of viruses can be used for the production of AAV, HSV with an in tact ICP27 gene is less effective than HSV in which ICP27 is deleted or mutated. This is presumably because the ICP27 produced from the in tact ICP27 gene inhibits splicing of AAV transcripts.

Where ICP27 is deleted, the use of cells in which the virus cannot replicate (BHK cells) provides greater yields of AAV than does the use of cells in which replication can it occur (B130 cells).

Therefore, where ICP27 is deleted using BHK cells, yields which are initially high reduce as MOI is reduced, as the input virus cannot replicate. Using cells where the virus can replicate (B130 cells) only low yields are generated at any MOI, presumably as splicing is inhibited by the ICP27 produced from the B130 cells.

The viruses with ICP27 mutated produce high yields of AAV on BHK cells where the virus can replicate, better than the viruses in which ICP27 is not altered presumably as inhibition of splicing is reduced. Moreover these high yields are maintained as the MOI reduces better than is the case with the ICP27 deleted viruses, presumably because yield is maintained through virus replication.

As has previously been shown to be the case, HSV can be used for the production of AAV vectors (e.g. Conway et al). We have shown that the use of a replication competent virus containing a mutated ICP27 with which inhibition of splicing is reduced allows enhanced production of AAV at low MOI as compared to HSV in which ICP27 is deleted or in which ICP27 is in tact. Use of HSV with mutated ICP27 therefore provides advantages for AAV production as reduced MOI can be used which will be advantageous for the scale up of HSV-based AAV production systems.

References
Conway et al., 1999, Gene Therapy 6, 986-993
Soliman et al., 1997, J. Virology 71, 9188-9197
Maclean et al., 1991. J Gen Virol. 72 (Pt 4):897-906
Balan et al., 1994, J Gen Virol. 75 (Pt 6):1245-58
Howard et al., 1998, Gene Ther. 5(8):1137-47

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1-27. (canceled)

28. A method of producing an AAV vector comprising:
(i) introducing into producer cells:
(a) a herpes virus which lacks a functional wild-type HSV ICP27 gene;
(b) a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27;
(c) AAV rep and cap genes; and
(d) an AAV vector sequence; and
(ii) isolating the AAV vector particles produced.

29. A method according to claim 28 wherein said herpes virus (a) comprises said nucleic acid (b).

30. A method according to claim 28 wherein said nucleic acid (b) is stably or transiently infected into said producer cells.

31. A method according to claim 28 wherein said AAV rep and cap genes (c) and/or said AAV vector sequence (d) are inserted into said herpes virus (a).

32. A method according to claim 31 wherein said AAV rep and cap genes (c) and/or said AAV vector sequence (d) are inserted into the UL43 locus, US5 locus or LAT locus of said herpes virus.

33. A method according to claim 28 wherein said AAV rep and cap genes and/or said AAV vector sequence (d) are stably or transiently transfected into said producer cells.

34. A method according to claim 30 wherein said producer cells are stably transfected prior to infection with said herpes virus (a).

35. A method according to claim 30 wherein said producer cells are transiently transfected before infection with said herpes virus (a).

36. A method according to claim 30 wherein said producer cells are transiently transfected after infection with said herpes virus (a).

37. A method according to claim 28 wherein the producer cells are BHK or Vero cells.

38. A method according to claim 28 wherein said herpes virus is HSV-1 or HSV-2.

39. Use according to claim 28 wherein said ICP27 protein is a functional equivalent of ICP27 from a non-HSV herpes virus.

40. A method according to claim 39 wherein said functional equivalent is mutated.

41. A method according to claim 28 wherein said ICP27 protein is a mutant HSV ICP27 protein.

42. A method according to claim 41 wherein the mutant protein is an HSV ICP27 protein comprising an R480H/V496I double mutation.

43. A method according to claim 28 wherein the herpes virus is a non-HSV herpes virus which further lacks its wild-type functional equivalent of the HSV ICP27 gene.

44. An AAV vector produced by a method of claim 28.

45. A pharmaceutical composition comprising an AAV vector according to claim 44 and a pharmaceutically acceptable carrier or diluent.

46. A method of producing a pharmaceutical composition comprising mixing an AAV vector according to claim 44 with a pharmaceutically acceptable carrier or diluent.

47. A method of producing a pharmaceutical composition comprising carrying out the method of claim 28 and formulating said isolated AAV vector particles with a pharmaceutically acceptable carrier or diluent.

48. A method of gene therapy comprising administering a therapeutically effective amount of an AAV vector according to claim 44 to a patient in need thereof.

49. A kit for producing an AAV vector comprising:
(a) a replication competent herpes virus which lacks a functional wild-type HSV ICP27 gene;
(b) a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27;
(c) AAV rep and cap genes;
(d) an AAV vector sequence; and optionally
(e) producer cells
wherein said nucleic acid (b), said AAV rep and cap genes (c) and/or said AAV vector sequence (d) are incorporated into said herpes virus (a), are present on separate plasmids or are stably integrated into said producer cells (e).

50. A replication competent herpes virus which (a) lacks a functional wild-type HSV ICP27 gene;
(b) comprises a nucleic acid encoding an ICP27 protein, or a functional equivalent thereof from a non-HSV herpes virus, which allows replication of said herpes virus to occur and which has a reduced ability to inhibit RNA splicing compared to wild-type HSV ICP27; and
(c) comprises AAV rep and cap genes.

51. A herpes virus according to claim 50 which further comprises an AAV vector sequence.

52. A herpes virus according to claim 50 wherein said AAV rep and cap inserted into the UL43 locus, US5 locus or LAT locus of said herpes virus.

53. A herpes virus according to claim 51 wherein said AAV vector is inserted into the UL43 locus, US5 locus or LAT locus of said herpes virus.
